(19) United States
(12) Patent Application Publication
(10) Pub. No.: US 2003/0027219 A1
(43) Pub. Date: Feb. 6, 2003

(54) Methods for depositing small volumes of protein fluids onto the surface of a substrate

(76) Inventors: Diane D. Ilsley, San Jose, CA (US); Douglas A, Amorese, Los Altos, CA (US); Michael P. Caren, Palo Alto, CA (US); Peter Tsang, San Francisco, CA (US)

Correspondence Address: Agilent Technologies, Inc., Legal Department, DL429, Intellectual Property Administration, P.O. Box 7599, Loveland, CO, 80537-0599, US

(21) Appl. No.: 09/919,643

(22) Filed: Jul. 31, 2001

(51) Int. Cl. G01N 33/53; G01N 33/542; B05D 1/32

(52) U.S. Cl. 435/7.9; 427/466

(57) ABSTRACT

Methods for efficiently depositing small quantities of a protein containing fluid onto the surface of a substrate are provided. A feature of the subject methods is that the deposition process does not substantially modulate the protein activity/functionality of the deposited fluid. In practicing the subject methods, a small volume of fluid containing the protein(s) of interest is front loaded into a thermal inkjet device. Next, a small quantity of the front loaded fluid is expelled onto the surface of a substrate. The subject methods find use in a variety of different applications where the deposition of small volumes of a fluid containing a protein of interest is desired.

### INTRODUCTION

1. Background of the Invention

Bioanalytical Microsystems, such as biosensors and assays in a microarray format or reaction chamber, often require the use of a technology that can dispense very small quantities (pico- and nanoliters) of solutions containing biomolecules. For maximum use, the deposition technology must be rapid, highly reproducible, and deposit solutions with precise placement onto a given solid support. Furthermore, the deposited biomolecules should retain their functionality/activity after the deposition process. Several recent papers have described the design and construction of devices for the production of microarrays of biomolecules onto a solid support.

In particular, Roda et al., Biotechniques (2000) 28:492-496, describe a method in which a conventional inkjet printer is used for the microdeposition of proteins. In this report, the black ink was removed from an HP ink cartridge and the cartridge was extensively washed with water. The cartridge was filled with the protein deposition solution using a microsyringe and sealed. The protein solution was then deposited onto a solid support and allowed to air dry, where it remained active for 1-2 weeks when stored at 4° C. While this method achieves precise deposition of small quantities of fluid that retain the desired protein functionality, it has certain disadvantages. One problem with this method is that a minimum of 2 mL of solution is needed to fill the cartridge. Unused sample can potentially be recovered, with the exception of 200-300 μl that remains in the print head. As such, this method is not very efficient in terms of sample waste, which can be significant with respect to rare or expensive samples. Other problems include the fact that only a single solution can be loaded at a time and that loading is done manually. Thus, this protocol for deposition of fluid onto a surface is not optimal.

Similarly, Deeg et al. in U.S. Pat. No. 5,338,688, describe a method of using bubble-jet technology for the metered application of an analytical liquid to a target. This disclosed method is based on the manufacture of disposable jet units containing the analytical liquid in prepacked form. A preloaded jet may be cost effective, but lacks flexibility. As such, this method has limitations.

Accordingly, there is continued interest in the development of new protocols for use in the deposition of fluids containing proteins onto a substrate surface. Of particular interest would be the development of a protocol that efficiently uses only small volumes of a protein containing sample, is capable of depositing the sample without substantially changing the protein activity or functionality of interest in the sample, and allows the flexibility to change the protein solution deposited and deliver multiple reagents simultaneously.

2. Relevant Literature

U.S. Patents disclosing the use of inkjet devices to dispense bio/chemical agents such as proteins and nucleic acids include: U.S. Pat. Nos. 4,877,745; 5,073,495; 5,200,051; 5,338,688; 5,474,796; 5,449,754; 5,658,802; 5,700,637; 5,751,839; 5,891,394; 5,958,342, 6,221,653, and 6,112,605. Also of interest is Roda et al., Biotechniques (2000) 28:492-496; Graves et al., Anal. Chem. (1998) 70: 5085-5092; and Yershov et al., Proc. Nat'l Acad. Sci. USA (1996) 93: 4913-4918.

### SUMMARY OF THE INVENTION

Methods for efficiently depositing small quantities of fluids containing a protein(s) onto the surface of a substrate are provided. A feature of the subject methods is that the deposition process does not substantially modulate the protein activity/functionality in the deposited fluid. In practicing the subject methods, a small volume of fluid containing the protein(s) of interest is front loaded into a thermal inkjet device. Next, a small quantity of the front loaded fluid is expelled onto the surface of a substrate. The subject methods find use in a variety of different applications where the deposition of small volumes of a fluid containing a protein of interest is desired.

### DEFINITIONS

The term “peptide” as used herein refers to polymers produced by amide formation between a carboxyl group of one amino acid and an amino group of another group, where the polymers have fewer than about 10 to 20 amino acid residues, i.e. amino acid monomeric units.

The term “polypeptide” as used herein refers to peptides with more than 10 to 20 residues.

The term “protein” as used herein refers to polypeptides of specific sequence of more than about 50 residues.

### DESCRIPTION OF THE SPECIFIC EMBODIMENTS

Methods are provided for efficiently depositing a quantity of a fluid containing protein(s) onto the surface of a solid support. In the subject methods, a thermal inkjet head is front loaded with a small volume of the fluid. Following front loading, the loaded head is then positioned in opposing relationship to, e.g. over, the target surface of the substrate or solid support. The temperature of the heating element of the inkjet head is then raised such that a bubble is formed at the surface of the heating element and a small quantity of the fluid sample is expelled from the head onto the surface. A feature of the subject methods is that the protein activity or functionality in the fluid volume is not substantially modulated, if at all, by the deposition process or the drying of the fluid on the surface. The subject methods find use in a variety of different applications where the efficient deposition of a fluid containing protein(s) is desired.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this invention belongs.

The subject invention is a method of efficiently depositing a quantity of a fluid sample that includes one or more proteins of interest onto the surface of a substrate. As such, the subject invention is directed to a method of depositing one or more proteins of interest onto the surface of substrate. A feature of the subject methods is that the deposition process employed in the subject methods does not substantially modulate the activity of the protein(s) in the fluid during deposition. In other words, the overall activity or functionality of one or more proteins of interest in the fluid sample is not substantially increased or decreased by the deposition process. This preservation of protein activity feature of the subject invention is accomplished despite the small volumes of fluid that are employed and the manner in which fluid is deposited in the subject methods, i.e., via thermal inkjet deposition.

The fluid that is deposited by the subject invention is a fluid that contains one or more proteins of interest, e.g., binding molecules such as antigens, antibodies, ligands, etc., enzymes, and the like. The concentration of the protein of interest in the fluid sample may vary, but typically is at least about 1 μg/ml, usually at least about 10 μg/ml and more usually at least about 50 μg/ml, where concentration may be as high as 200 μg/ml, but typically does not exceed about 500 μg/ml and more typically does not exceed about 1000 μg/ml. The fluid sample may be prepared in a number of different manners, e.g., it may be derived from a naturally occurring fluid that may have been processed in one or more ways, e.g., to enrich or purify the protein of interest, it may be the product of a synthetic process, e.g., automated protein synthesis procedure, and the like.

As indicated above, a feature of the subject methods is the use of a thermal inkjet head to deposit a quantity of the fluid sample onto the substrate surface. Thermal inkjet heads are well known in the art of conventional printing and document production. As is known to those of skill in the art, thermal inkjet heads typically have at least the following components: (a) an orifice; (b) a firing chamber; and (c) a heating element. Thermal inkjet heads and methods for their manufacture and use are described in a number of different U.S. Patents, including: U.S. Pat. Nos. 5,772,829; 5,745,128; 5,736,998; 5,736,995; 5,726,690; 5,714,989; 5,682,188; 5,677,577; 5,642,142; 5,636,441; 5,635,968; 5,635,966; 5,595,785; 5,477,255; 5,434,606; 5,426,458; 5,350,616; 5,341,160; 5,300,958; 5,229,785; 5,187,500; 5,167,776; 5,159,353; 5,122,812; and 4,791,435; the disclosures of which are herein incorporated by reference.

Thermal inkjet heads finding use in the subject methods will generally have the following characteristics. The size of the orifice is sufficient to produce a spot of suitable dimensions on the substrate surface (described in greater detail infra), where the orifice generally has a diameter (or exit diagonal depending on the specific format of the ink jet head) ranging from about 1 to 1000 μm, usually from about 5 to 100 μm and more usually from about 10 to 60 μm. The filing chamber has a volume ranging from about 1 pl to 10 nl, usually from about 10 pl to 5 nl and more usually from about 35 pl to 1.5 nl. The heating element will preferably be made out of a material that can deliver a quick energy pulse, where suitable materials include: TaAl and the like. The thermal element is capable of achieving temperatures sufficient to vaporize a sufficient volume of the nucleic acid composition in the firing chamber to produce a bubble of suitable dimensions upon actuation of the head. Generally, the heating element is capable of attaining temperatures of at least about 100° C., usually at least about 400° C. and more usually at least about 700° C., where the temperature achievable by the heating element may be as high as 1000° C. or higher. The device may also have one or more reservoirs. In other words, the device may be a single reservoir device or a multi-reservoir device. When present, the reservoir will typically have a volume ranging from about 1 pl to 1 l, usually from about 0.5 μl to 10 μl and more usually from about 1 μl to 5 μl. A variety of thermal inkjet heads are available commercially, where such devices include: the HP92261A thermal inkjet head (available from Hewlett-Packard Co., Palo Alto Calif.), the HP 51645A thermal inkjet head (available from Hewlett-Packard Co. Palo Alto Calif.), the inkjet produced by (Cannon Kabushiki Kaisha, Tokyo, Japan) and the like. Specific inkjet heads of interest are disclosed in U.S. Pat. Nos. 5,736,998 and 4,668,052, the disclosures of which are herein incorporated by reference.

In practicing the subject methods, the thermal inkjet device is front loaded with a fluid sample containing the one or more proteins of interest. Because the methods are methods of efficiently depositing a volume or quantity of fluid onto a surface, such that the amount of fluid required is small and most efficiently and effectively utilized, a front loading procedure is typically employed for loading the fluid into the head. In this front loading protocol, the orifice is contacted with the fluid under conditions sufficient for fluid to flow through the orifice and into the firing chamber of the head, where fluid flow is due, at least in part, to capillary forces. To assist in the flow of fluid into the orifice, back pressure in the form of suction (i.e. negative pressure) may be applied to the firing chamber (and reservoir, if present) of the head, where the back pressure will typically be at least about 5, and may be at least about 10 and even as great as about 100 inches of H₂O or more.

The amount of fluid required to load the head is typically small, generally not exceeding more than about 10 μl, usually not exceeding more than about 5 μl and in many embodiments not exceeding more than about 2 μl. As such, the amount of fluid that is wasted in readying or preparing the thermal inkjet head for firing is minimal. As such, fluid loading is highly efficient. Therefore, the subject methods are particularly suited for use with rare and/or expensive fluid samples.

Following front loading of the inkjet head, the head is employed to deposit an extremely small quantity of a fluid sample, e.g., a pico liter volume of fluid sample, onto the surface of a substrate. As the subject methods are capable of depositing an extremely small volume of fluid onto a substrate surface, the subject methods can be used to deposit a pico liter quantity of fluid onto an array surface. By “pico liter quantity” is meant a volume of fluid that is at least about 0.1 pl, usually at least about 1 pl and more usually at least about 10 pl, where the volume may be as high as 250 pl or higher, but generally does not exceed about 100 nL and usually does not exceed about 1 μl.

As indicated above, a feature of the subject methods is that the deposition process does not result in a substantial modulation of the activity or functionality of the protein(s) of interest in the fluid that is deposited, despite the small volumes front loaded into the head and the thermal inkjet deposition protocol employed. In other words, the overall protein activity/functionality of interest in the fluid that is deposited from the inkjet during the subject methods is not substantially different from the overall protein activity in the fluid loaded into the inkjet prior to deposition. As such, the protein activity of a quantity of fluid deposited from the inkjet is substantially the same as that of an identical amount of fluid still present in the inkjet. In other words, use of the subject methods to deposit the fluid onto the surface does not adversely affect the desired protein activity/functionality of the protein of interest in the fluid.

More specifically, if the sum of all of the individual activities of the individual protein molecules in the deposited volume of fluid is viewed as the overall protein activity of the fluid for the deposited volume of fluid, then the deposition process does not substantially change the overall protein activity of the deposited fluid sample, if at all, because the deposition process does not modify a significant percentage of the total number of protein molecules present in the deposited fluid sample. Since a significant percentage of the total number of protein molecules in the quantity of deposited fluid is not modified by deposition according to the subject methods, the total percentage of protein molecules that are modified, e.g., denatured, degraded or otherwise inactivated etc., at least partially or completely, by the deposition process does not exceed about 10%, usually does not exceed about 5% and more usually does not exceed about 1%. For example, where a given quantity of deposited fluid contains 1000 identical antibody molecules, deposition results in degradation or denaturation, either partially or completely, of less than 100 of these molecules, usually less than 50 of these molecules and more usually less than 10 of these molecules, if any. In terms of concentration of the active protein of interest, any change in concentration of the activity or function protein of interest in the sample that occurs in the deposited fluid does not exceed about 20%, usually does not exceed about 10% and more usually does not exceed about 5%.

In terms of the overall protein activity, the amount of modulation, if any, that occurs because of the manner of deposition is typically less than about 10%, usually less than about 5% and more usually less than about 1%. A convenient means of determining the amount of change in overall protein activity caused by deposition is to compare the protein activity of a quantity of fluid that has been expelled or fired from the inkjet to the protein activity of the same quantity of an identical fluid that has not been expelled or fired, e.g., loaded fluid still in the head. The particular assay that is employed to achieve the above comparison necessarily varies depending on the particular nature of the protein and activity/functionality of interest.

In certain embodiments, the protein activity that is preserved during the subject methods is the overall protein binding activity of the protein in the sample of interest, specifically, the sum of the individual binding activities of the protein molecules in the sample. Thus, where the protein of interest is a member of a specific binding pair, e.g., an antibody or antigen, the overall binding activity or binding functionality of the deposited fluid made up of the sum of the binding activity of all of the protein molecules of interest in the fluid is not substantially modulated by the deposition process. As such, any modulation or change, generally decrease, in the overall binding activity or functionality of the deposited fluid quantity does not exceed about 10%, usually does not exceed about 5% and more usually does not exceed about 1%.

In certain embodiments, the protein activity or functionality that is preserved in the deposited quantity of fluid is an enzymatic activity. In these embodiments, any change in activity, e.g., decrease, in enzymatic activity that is observed in the deposited fluid as compared to the predeposited fluid is not substantial, such that it does not exceed about 10%, usually does not exceed about 5% and more usually does not exceed about 1%.

As such, the fluid loading and deposition process of the subject invention is extremely efficient in that waste is substantially eliminated and only small volumes of fluid are required for loading of the fluid into the head, making the subjects methods particularly suited for use with rare and/or valuable protein fluids.

In the broadest sense, the subject methods may be used to deposit a volume of fluid sample onto any structure, specifically a surface, of any substrate, where the substrate may be a planar structure, e.g., a slide, a reagent container, e.g., a well in a multiwell plate (such as the bottom of a well), a channel or micro structure, an array etc.

To deposit fluid onto the substrate surface according to the subject methods, the loaded thermal inkjet head is positioned in opposing relationship relative to the surface of the substrate (e.g. with an XYZ translational means), where the orifice is in opposition to the position on the array surface at which deposition of the protein solution is desired (e.g. opposite a binding agent spot on the array). The distance between the orifice and the substrate surface will not be so great that the volume of protein fluid cannot reach the substrate surface and produce a spot in a reproducible manner. As such, the distance between the orifice and the substrate surface will generally range from about 10 μm to 10 mm, usually from about 100 μm to 2 mm and more usually from about 200 μm to 1 mm.

After the head is placed into position relative to the substrate surface, the temperature of the heating element or resistor of the head is raised to a temperature sufficient to vaporize a portion of the fluid immediately adjacent to the resistor and produce a bubble. In raising the temperature of the heating element, the temperature of the heating element is raised to at least about 100° C., usually at least about 400° C. and more usually at least about 700° C., where the temperature may be raised as high as 1000° C. or higher, but will usually be raised to a temperature that does not exceed about 2000° C. and more usually does not exceed about 1500° C. As such, a sufficient amount of energy will be delivered to the resistor to produce the requisite temperature rise where the amount of energy will generally range from about 1.0 to 100 μJ, usually from about 1.5 to 15 μJ. The portion of fluid in the firing chamber that is vaporized will be sufficient to produce a bubble in the firing chamber of sufficient volume to force an amount of liquid out of the orifice.

The formation of the bubble in the firing chamber traps a portion or volume of the fluid present in the firing chamber between the heating element and the orifice and forces an amount or volume of fluid out of the orifice at high speed. The amount or volume of fluid that is forced out of the firing chamber can be controlled depending on the specific amount of fluid that is desired to be deposited on the substrate. As is known in the art, the amount of fluid that is expelled can be controlled by changing one or more of a number of different parameters of the ink jet head, including: the orifice diameter, the orifice length (depth), the size of the firing chamber, the size of the heating element, and the like. Such variations are well known to those of skill in the art. As such, the amount or volume of fluid that is forced out or expelled from the firing chamber may range from about 0.1 to 2000 pl, usually from about 0.5 to 500 pl and more usually from about 1.0 to 250 pl. The speed at which the fluid is expelled from the firing chamber is at least about 1 m/s, usually at least about 10 m/s and may be as great as about 20 m/s or greater.

Upon actuation of the thermal inkjet head, as described above, fluid is expelled from the orifice and travels to the substrate surface. Upon contact with the substrate surface, the deposited fluid typically forms a spot on the substrate surface. As mentioned above, by varying the design parameters of the thermal inkjet head, the spot dimensions can be controlled such that spots of various sizes can be produced. With the subject methods, one can produce spot sizes which have diameters ranging from a minimum of about 10 μm to a maximum of about 1.0 cm. In those embodiments where very small spot sizes are desired, one can produce small spots that have a diameter ranging from about 1.0 μm to 1.0 mm, usually from about 5.0 μm to 500 μm and more usually from about 10 μm to 200 μm. In many embodiments, the spot sizes range from about 30 to 100 μm in diameter.

As indicated above, an important feature of the subject invention is that the deposition process does not adversely affect the overall protein activity or functionality of the protein of interest in the sample, despite the small amount of fluid that is loaded into the head and then expelled from the head.

In certain embodiments, it may be desirable to prevent evaporation of the fluid sample following deposition. Evaporation may be prevented in a number of different ways. The subject methods may be carried out in a high humidity environment. By “high humidity” is meant an environment in which the humidity is at least about 86% relative humidity, usually at least about 95% relative humidity and more usually at least about 99% relative humidity. Alternatively, one may apply an evaporation retarding agent, e.g. mineral oil, glycerol solution, polyethylene glycol, etc., over the surface of the deposited sample, e.g. by using a thermal inkjet as described above.

In yet other embodiments, it may be desired to rapidly dehydrate the deposited sample following deposition, e.g., where it is desired to produce a dry deposited sample on the substrate surface, e.g., for storage prior to use. By depositing the fluid sample in a dry environment and a suitable temperature, the water component of the deposited fluid sample rapidly evaporates, leaving active protein that can be readily stored for subsequent use. In these embodiments, the relative humidity of the environment typically does not exceed about 35%, usually does not exceed about 20% and more usually does not exceed about 10%. The temperature typically ranges from about 2° C. to about 30° C., usually from about 4° C. to about 25° C. and more usually from about 10° C. to about 20° C.

Where desired, following deposition of the desired amount of protein fluid, the head may be washed and front loaded with another protein containing fluid for subsequent fluid deposition. Washing of the head can be accomplished using any convenient protocol, e.g., via front loading and expelling an appropriate wash buffer, one or more times, by backloading and expelling an appropriate wash buffer, etc. In addition, the head may be manually or automatically wiped clean to remove any sample/wash solution left from the previous deposition.

In many embodiments, the head is rapidly washed and reloaded with a new solution, such that the time period starting from the deposition of the first fluid to the loading of the second fluid, i.e., the washing time, is extremely short. In these embodiments, the wash time typically does not exceed about 1 minute, usually does not exceed about 5 minutes and more usually does not exceed about 30 minutes. The wash protocol in these embodiments may include a single flushing or multiple flushes, where the total number of flushes will typically not exceed about 3, usually will not exceed about 5 and more usually will not exceed about 10. The wash fluid employed in these embodiments is typically one that provides for removal of substantially all proteins of the first fluid in a minimal number of flushes, where representative fluids of interest include, but are not limited to: saline buffer solution with surfactent, and the like.

The above methods can be substantially, if not completely automated, so that fluid can be loaded and deposited onto a surface automatically. As such, the subject methods are amenable to high throughput applications, e.g., high throughput manufacturing applications. In automated versions of the subject methods, automated devices are employed that are analogous to conventional thermal inkjet printing devices, with the exception that the thermal inkjet head of the device is front loaded with a fluid sample as described above, instead of ink. Such automatic devices comprise at least a means for precisely controlling the position of the head with respect to an array surface (an XYZ translational mechanism) and for firing the head. Such automated devices are well known to those of skill in the printing and document production art, and are disclosed in U.S. Pat. Nos. 5,772,829; 5,745,128; 5,736,998; 5,736,995; 5,726,690; 5,714,989; 5,682,188; 5,677,577; 5,642,142; 5,636,441; 5,635,968; 5,635,966; 5,595,785; 5,477,255; 5,434,606; 5,426,458; 5,350,616; 5,341,160; 5,300,958; 5,229,785; 5,187,500; 5,167,776; 5,159,353; 5,122,812; and 4,791,435; the disclosures of which are herein incorporated by reference.

The subject methods of depositing a volume of fluid sample onto the surface of a substrate find use in a variety of different applications, and are particularly suited for use in methods where reproducible placement of small volumes of a reagent onto the surface of a solid support are desired. As such, the subject methods find use in the preparation and manufacture of biosensors, microarrays, e.g., proteomic arrays, microfluidic devices, and the like.

In the course of practicing the subject methods, fluid contacted arrays are produced in which each deposited fluid volume occupies a small area, i.e. spot, on the substrate surface. By small is meant that each fluid sample spot on the array has a diameter that is at least about 1 μm, usually at least about 5 μm and more usually at least about 10 μm and does not exceed about 10 mm, usually does not exceed about 1000 μm and more usually does not exceed about 200 μm. An important feature of the subject methods is that the deposited protein retains its activity or functionality.

The subject arrays produced in accordance with the invention find use in a variety microarray applications, including analyte detection applications in which the presence of a particular analyte in a given sample may be detected. Protocols for carrying out such assays are well known to those of skill in the art and need not be described in detail herein. Briefly, a sample comprising the analyte of interest is contacted with an array produced according to the subject methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g. through use of a signal production system, e.g. an isotopic or fluorescent label present on the analyte, etc. The presence of the analyte in the sample is then deduced from the detection of binding complexes on the substrate surface. Specific applications of interest include analyte detection/proteomics applications, including those described in: U.S. Pat. Nos. 4,591,570; 5,171,695; 5,436,170; 5,486,452; 5,532,128; and 6,197,599; the disclosures of which are herein incorporated by reference; as well as published PCT application Nos. WO 99/39210; WO 00/04832; WO 00/04389; WO 00/04390; WO 00/54046; WO 00/63701; WO 01/14425; and WO 01/40803; the disclosures of the United States priority documents of which are herein incorporated by reference.

In certain embodiments, the subject methods include a step of transmitting data from at least one of the detecting and deriving steps, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet etc.

As such, in using an array made by the method of the present invention, the array will typically be exposed to a sample (for example, a fluorescently labeled analyte, e.g., protein containing sample) and the array then read. Reading of the array may be accomplished by illuminating the array and reading the location and intensity of resulting fluorescence at each feature of the array. For example, a scanner may be used for this purpose which is similar to the GENEARRAY scanner available from Agilent Technologies, Palo Alto, Calif. Other suitable apparatus and methods are described in U.S. patent application Ser. No. 09/846125 “Reading Multi-Featured Arrays” by Dorsel et al.; and Ser. No. 09/430214 “Interrogating Multi-Featured Arrays” by Dorsel et al. As previously mentioned, these references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

The following examples are offered by way of illustration and not by way of limitation.

### Experimental

### EXAMPLE I

Using a XYZ motion system and Hewlett Packard inkjet head (HP#516454), 2 μl of 100 μg/ml bovine serum albumin (BSA) in 50 mM Tris-HCl, pH 7.5, 50 mM NaCl, and 0.05% SDS was front loaded into 6 reservoirs of the inkjet. The concentration of BSA solution was determined using Bradford Reagent. The head was fired multiple times, and the solution was collected for analysis (6 μl). 2 μl of the pre- and post-fired solutions were analyzed using the Caliper prototype protein LabChip™ assay system. The resultant overlayed electropherograms showed that the BSA was not degraded during the firing of the inkjet and that the protein concentration of the pre- and post-fired solutions were essentially equivalent. The concentration of the first load of BSA into the inkjet was slightly lower than the pre-fired sample and the second loaded BSA solution, indicating that a small amount of protein did bind irreversibly in the head the first time protein was loaded. However, with subsequent loading and firing of protein of solutions, the pre- and post-fired solutions were essentially equivalent.

### EXAMPLE II

A mouse monoclonal antibody to dsDNA (500 μg/ml total protein) was loaded into 6 reservoirs of the inkjet, fired multiple times, and collected (10 μl). 2 μl were analyzed using the Caliper prototype protein LabChip™ assay system. The resultant spectrums of the pre- and post-fired solutions were identical. Functionality of the antibody was determined by its ability to bind to dsDNA. The pre- and post-fired antibody solutions (8 μl) were incubated in 250 μl NETG buffer (150 mM NaCl, 5 mM EDTA, 48 mM Tris-HCl, pH 7.4, 0.25% gelatin) on a cDNA microarray at room temperature for 1 hour. The slide was then washed once with 1×NETG and 3× with 1/100×NETG. A secondary antibody, goat anti-mouse conjugated to rhodamine, was added and incubated for 1 hour at room temperature to detect binding of the primary antibody to dsDNA. The slide was washed as described above, and scanned on the Axon scanner. The results demonstrated that pre- and post-fired antibody solutions gave similar fluorescent signals and specificity for binding the DNA. Firing the antibody through the inkjet did not appear to affect functionality.

### EXAMPLE III

To demonstrate that proteins can be readily removed from the head by washing, a solution of 5 proteins (Mr=16,000; 97,000; 66,000; 45,000; and 18,500) of known concentration are loaded into the head, fired and collected. The head is washed and loaded with buffer. The buffer is fired and collected. The concentration of the pre- and post-fired protein and buffer solutions is determined using the Caliper protein LabChip™ assay system. The analysis shows that the pre- and post-fired protein solutions are indistinguishable, and there is no detectable protein in the post-fire buffer solution.

### EXAMPLE IV

A glass slide containing inkjet deposited cDNAs that are crosslinked to the surface is used. Cy5-dCTP is had spotted randomly onto the surface and allowed to dry. A solution containing buffer and dNTPs and a second solution containing DNA polymerase is loaded into an inkjet and fired onto the glass slide. The slide is incubated in a humid chamber at 37° C. for 60 minutes to allow DNA polymerization. The slide is washed to remove unincorporated Cy5-dCTP. The slide is then scanned for covalently linked Cy5-dCMP to the DNA attached to the surface, indicating that the DNA polymerase synthesized DNA. The results show that multiple reagents may be deposited onto the surface using the subject methods. The results also show that the activity of the polymerase enzyme is maintained during the deposition process.

### EXAMPLE V

Monoclonal antibody solutions with total protein concentrations of 50 μg/mL, 100 μg/mL, 200 μg/mL, 500 μg/mL, and 1000 μg/mL in 50 mM NaCl, 50 mM Tris-HCl, pH 7.5, 0.05% SDS. (most of the protein is BSA) were sequentially loaded into the inkjet head. The inkjet head was loaded with the first solution (50 μg/mL). The head was fired multiple times and the solution fired was collected (about 4-5 μL). The same solution was loaded again, fired, and collected. The head was then washed 6 times with 3 mL sodium phosphate buffer, pH 7.4, followed by 3 mL water. Then the next concentration was loaded, fired, and collected, and the head was washed. This process was continued until the 1000 μg/mL solution was fired, where after several fires, the inkjet head died.

It is evident from the above results and discussion that a simple and efficient way to deposit protein containing fluid samples onto surfaces is provided by the subject invention. The subject methods are highly efficient in that only small volumes of the protein fluid are required and fluid waste is substantially eliminated. As such, the subject methods are particularly suited for use with rare and/or valuable protein containing fluid samples. In addition, the subject methods produce reproducible spots are amenable to automation, therefore making them particularly suited for use in high throughput applications, including high throughput manufacturing applications. As such, the subject invention is a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method for depositing a quantity of a fluid containing a protein of interest onto a surface of a substrate, said method comprising:
(a) front loading said fluid into a thermal inkjet head comprising an orifice and a firing chamber by contacting said orifice with said fluid in a manner sufficient for said fluid to flow through said orifice into said firing chamber;
(b) positioning said loaded thermal inkjet head in opposing relation to said surface; and
(c) actuating said thermal inkjet head to deposit said quantity of fluid onto said surface.

2. The method according to claim 1, wherein said method further comprises applying back pressure to said head during said contacting step.

3. The method according to claim 2, wherein no more than about 5 μl of fluid is loaded into said head during said loading step.

4. The method according to claim 3, wherein no more than about 2 μl of fluid is loaded into said head during said loading step.

5. The method according to claim 1, wherein said protein of interest is present in said fluid at a concentration that ranges from about 5 to 1000 μg/ml.

6. The method according to claim 1, wherein said method further comprises washing said head following said actuating step (c).

7. The method according to claim 1, wherein said protein of interest is a member of a specific binding pair.

8. The method according to claim 1, wherein said protein of interest is an enzyme.

9. The method according to claim 1, wherein said surface is a surface of a planar substrate.

10. The method according to claim 1, wherein said surface is a surface of a reagent chamber.

11. The method according to claim 1, wherein said deposited quantity does not exceed about 200 picoliters.

12. A method for depositing a quantity of fluid containing a protein binding pair member onto a substrate surface, said method comprising:
(a) front loading less than about 5 μl of said fluid into a thermal inkjet head comprising an orifice and a firing chamber by contacting said orifice with said fluid and applying back pressure to said head during said contacting in a manner sufficient for said fluid to flow through said orifice into said firing chamber;
(b) positioning said loaded thermal inkjet head loaded with said fluid in opposing relation to said surface;
(c) actuating said thermal inkjet head to deposit said quantity of fluid onto said surface; and
(d) washing said head.

13. The method according to-claim 12, wherein no more than about 2 μl of fluid is loaded into said head during said loading step.

14. The method according to claim 12, wherein said protein binding pair member is present in said fluid at a concentration ranging from about 5 to 1000 μg/ml.

15. The method according to claim 12, wherein said surface is a surface of a planar support.

16. The method according to claim 12, wherein said surface is a surface of a reagent chamber.

17. A method for depositing a quantity of fluid containing an enzyme onto a surface of a substrate, said method comprising:
(a) loading less than about 5 μl of said fluid into a thermal inkjet head comprising an orifice and a firing chamber by contacting said orifice with said fluid and applying back pressure to said head during said contacting in a manner sufficient for said fluid to flow through said orifice into said firing chamber;
(b) positioning said loaded thermal inkjet head loaded with said fluid in opposing relation to said surface;
(c) actuating said thermal inkjet head to deposit said quantity of fluid onto said surface; and
(d) washing said head.

18. The method according to claim 17, wherein no more than about 2 μl of fluid is loaded into said head during said loading step.

19. The method according to claim 17, wherein said enzyme is present in said fluid at a concentration ranging from about 5 to 1000 μg/ml.

20. The method according to claim 17, wherein said surface is a surface of a planar substrate.

21. The method according to claim 17, wherein said surface is a surface of a reagent chamber chamber.

22. A method for depositing a quantity of a fluid containing a protein of interest onto a surface of a substrate, said method comprising:
(a) loading said fluid into a thermal inkjet head comprising an orifice and a firing chamber, wherein said protein of interest is present in said fluid at a concentration that ranges from about 5 to 1000 μg/ml;
(b) positioning said loaded thermal inkjet head in opposing relation to said surface; and
(c) actuating said thermal inkjet head to deposit said quantity of fluid onto said surface.

23. The method according to claim 22, wherein said method further comprises washing said head following said actuating step (c).

24. The method according to claim 22, wherein said protein of interest is a member of a specific binding pair.

25. The method according to claim 22, wherein said protein of interest is an enzyme.

26. The method according to claim 22, wherein said surface is a surface of a planar substrate.

27. The method according to claim 22, wherein said surface is a surface of a reagent chamber.

28. The method according to claim 1, wherein said deposited quantity does not exceed about 200 picoliters.

29. A surface produced by the process of claim 1.

30. The surface according to claim 29, wherein said surface is the surface of an array.

31. In a method of performing an assay employing a microarray, the improvement comprising:
employing an array according to claim 30.

32. A method of detecting the presence of an analyte in a sample, said method comprising:
contacting (a) a polymeric array according to claim 30 having a polymeric ligand that specifically binds to said analyte, with (b) a sample suspected of comprising said analyte under conditions sufficient for binding of said analyte to a polymeric ligand on said array to occur; and
detecting the presence of binding complexes on the surface of the said array; whereby the presence of said analyte in said sample is detected.

33. The method according to claim 32, wherein said method further comprises a data transmission step.

34. A kit for use in an assay that employs an array, said kit comprising:
an array according to claim 28; and
instructions for using said array in a hybridization assay.
